(19) United States
(12) Patent Application Publication
(10) Pub. No.: US 2022/0157441 A1
(43) Pub. Date: May 19, 2022

(54) REWARD HYGIENE SYSTEM

(71) Applicant: Essity Hygiene and Health Aktiebolag, Göteborg (SE)

(72) Inventor: Håkan LINDSTRÖM, GÖTEBORG (SE)

(73) Assignee: Essity Hygiene and Health Aktiebolag, Göteborg (SE)

(21) Appl. No.: 17/439,469

(22) PCT Filed: Apr. 2, 2019
(86) PCT No.: PCT/EP2019/058259

§ 371 Date: Sep. 15, 2021

(51) Int. Cl. G16H 40/20; G08B 21/24

(52) U.S. Cl. CPC G16H 40/20; G08B 21/245

(57) ABSTRACT

A monitoring system for hygiene equipment, the system comprising an interface configured to receive information indicating an instance of using a piece of hygiene equipment; a determination section configured to determine whether the instance is a compliant instance to use the piece of hygiene equipment; a storage section configured to store information on past compliance; a selector configured to select whether the compliant instance is to result in a reward action; and an adjusting section configured to adjust the selector based on the stored information on the past compliance.

### TECHNICAL FIELD

The present disclosure generally relates to monitoring the use of hygiene equipment by detecting an instance of using a piece of hygiene equipment which may be brought in relation to an opportunity to use said piece of hygiene equipment. More specifically, the present invention relates to a monitoring system that is to promote the use of hygiene equipment in the appropriate or desired way.

### BACKGROUND

Hygiene equipment is commonplace today in many facilities, such as hospitals, medical service centers, intensive care units, day clinics, private practices, lavatories, rest rooms, hotels, manufacturing sites, administration and office buildings, or, in general, places and facilities that are accessible to the public or to a considerable number of individuals where a certain level of hygiene ought to be maintained. The mentioned hygiene equipment thereby includes various types of individual devices and installations such as soap dispensers, dispensers for disinfectant solutions, gels or substances, towel dispensers, glove dispensers, tissue dispensers, hand dryers, sinks, radiation assisted disinfectant points, and the like.

Although such hygiene equipment is commonplace today in many places, the use thereof by the individuals visiting these places or working in these places is still oftentimes not satisfactory. For example, hospitals, private practices, or, in general, medical service centers often suffer from hygiene deficiencies, which, in turn, may lead to the spread of infections and related diseases. In particular, such insufficient hygiene amongst medical care personnel coming into close contact with patients and bodily fluids can lead to the spread of infectious diseases amongst the personnel and other patients. It is also known that infections by highly resistant bacteria pose a severe problem in such places, especially in hospitals. In general, so-called Healthcare Associated Infections (HAI) are a real and tangible global problem in today's healthcare. HAI can be found to be currently the primary cause of death for 140.000 patients/year, affecting millions more and costs society in the range of billions of EUR/year.

At the same time, however, it is known that hygiene, and, in particular, hand hygiene, is an important factor as far as the spread of infectious diseases are concerned. Specifically, medical care personnel should make proper use of hand hygiene as often as possible so that the spread of bacteria and other disease causing substances is minimized. The actual usage of such hygiene equipment, however, may depend on—amongst others—the management of the facility, accessibility and usability of the equipment, culture, the cooperation and will exercised by the individuals working in these places or visiting such places, training of individuals, time pressure and possibly also other factors. In other words, an important factor remains the fact that individuals may not make use of installed and provided hygiene equipment although they are supposed to. Furthermore, it is generally accepted that an increased use of hygiene equipment can substantially contribute in reducing the spread of bacteria and the like, which, in turn, can drastically reduce the appearance of related infections and diseases.

For example, one can speak of a relatively low compliance which then may indicate that the actual use of hygiene equipment is not satisfactory, whilst a relatively high compliance may indicate that the actual use of hygiene equipment corresponds, within a given threshold, to some target usage, and, consequently, may be regarded as being satisfactory. Such compliance can be measured as a metric in the form of a value and may provide many advantages, since it gives a concise picture to operators of the corresponding facility so that they may initiate measures for increasing the actual use of hygiene equipment.

Therefore, there are already ways of estimating such a compliance metric in the arts, wherein the conventional approaches usually rely on measuring and/or observe “manually”, i.e. by a human observer, so-called opportunities and comparing these obtained opportunities to a measured/detected/observed actual use of the hygiene equipment. In other words, the opportunities indicate any event when hygiene equipment should and/or could have been used. By then comparing this “should/could”-value to an actual usage value a compliance metric can be calculated, as, e.g., a percentage value or a ratio. In general, the opportunities can be well defined figures, since they may be associated to specific rules and/or recommendations. For example, the World Health Organization (WHO) has defined the so-called “My 5 Moments Of Hand Hygiene” (see www.who.int/psc/tools/Five_moments/en/) including as explicit definitions for opportunities: 1. before touching a patient; 2. before clean/aseptic procedures; 3. after body fluid exposure/risk; 4. after touching a patient; and 5. after touching patient surroundings. Moreover, measurements on a corresponding hand hygiene compliance is becoming a regulatory requirement for the healthcare sector and may serve as an important quality improvement tool.

As a consequence, one may have considerable interest in that a given or desired target compliance is achieved. More specifically, it may be desired that across an entire facility, a high level of compliant hand hygiene is to be achieved and maintained. In other words, there is considerable interest that the individuals (operators) that are involved with such facilities implement the hygiene scheme as good as possible so as to reduce the spread of any disease as effectively as possible. At the same time, however, the actual use of hygiene equipment may quite considerably depend on the cooperation shown by the individuals or groups of individuals (operators in different departments).

There is therefore a need for improved systems and methods of promoting the use of hygiene equipment so that preferably a target use compliance is achieved and the spread of diseases is substantially reduced, whilst the implementation of the system should interfere as little as possible with existing surroundings, such as the mentioned hospitals, wards, and related facilities.

### SUMMARY

Embodiments of the disclosure solve the technical problems mentioned above.

According to one aspect of the present invention, there is provided a monitoring system for hygiene equipment, the system comprising an interface configured to receive information indicating an instance of using a piece of hygiene equipment; a determination section configured to determine whether the instance is a compliant instance to use the piece of hygiene equipment; a storage section configured to store information on past compliance; a selector configured to select whether the compliant instance is to result in a reward action; and an adjusting section configured to adjust the selector based on the stored information on the past compliance.

According to one aspect of the present invention, there is provided a method for operating a system that monitors hygiene equipment comprising the steps of receiving information indicating an instance of using a piece of hygiene equipment; determining whether the instance is a compliant instance to use the piece of hygiene equipment; storing information on past compliance; selecting with an selector whether the compliant instance is to result in a reward action; and a step of adjusting the selector based on the stored information on the past compliance instances.

### BRIEF DESCRIPTION OF THE DRAWINGS

Embodiments of the present invention, which are presented for better understanding the inventive concepts but which are not to be seen as limiting the invention, will now be described with reference to the figures in which:

FIGS. 1A and 1B show schematic views of monitoring systems for hygiene equipment according to respective general embodiments of the present invention;

FIG. 2 shows a schematic view of a deployment example of an embodiment of the present invention;

FIGS. 3A to 3E show schematic views of further pieces of hygiene equipment according to corresponding further embodiments of the invention;

FIG. 4 shows a schematic view of a system coupled to an indoor positioning system according to another embodiment of the present invention.

FIG. 5 shows a schematic view of a general apparatus embodiment of the present invention; and

FIG. 6 shows a schematic view of a general method embodiment of the present invention.

### DETAILED DESCRIPTION

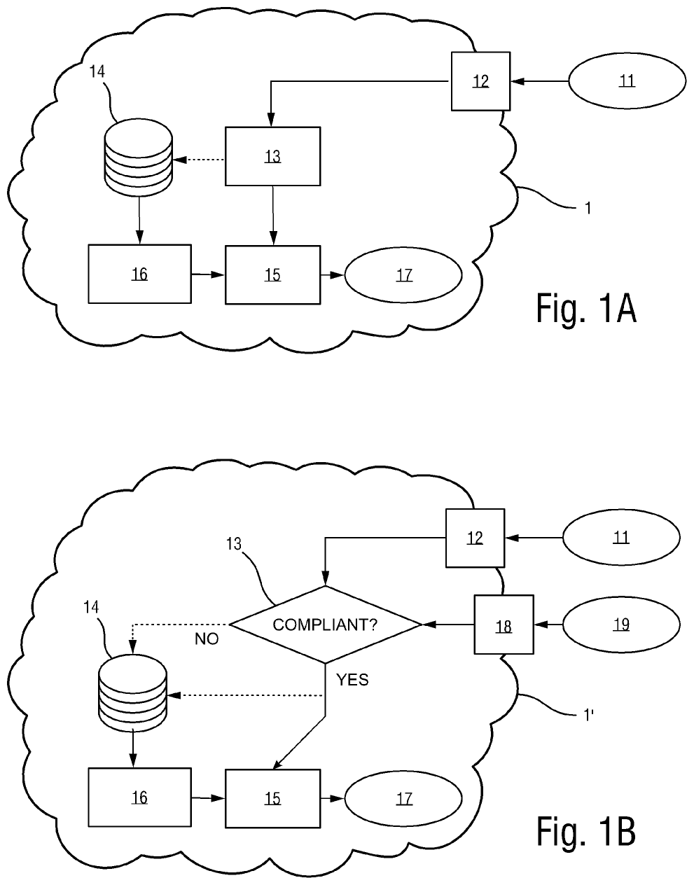

FIG. 1A shows a schematic view of a monitoring system for hygiene equipment according to a general embodiment of the present invention. According to this embodiment there is provided a monitoring system 1 for hygiene equipment, which comprises an interface 12 that is configured to receive information indicating an instance of using a piece of hygiene equipment. This information can be provided in the form of a message or signal 11 that can be received or obtained by the interface 12. The system further comprises a determination section 13 that is configured to determine whether the instance is a compliant instance to use the piece of hygiene equipment and a storage section 14 that is configured to store information on past compliance. For example, all or some instances can be recorded in the storage section 14 for later retrieval or analysis. The system 1 further comprises a selector 15 which is configured to select whether the compliant instance is to result in a reward action. The reward action can be initiated by means of a further signal or message 17 and the details in the context of the reward action as such are given elsewhere in the present disclosure. Yet further, the system 1 comprises an adjusting section 16 which is configured to adjust the selector based on the stored information on the past compliance instances. As will be detailed further below, the latter adjustment may depend on and consider one or more of specific factors obtained or evaluated by the system.

In one embodiment, if for some time period a certain number of compliant instances have been received, and in the same time another certain number of opportunities for using the hygiene equipment have been received a compliance (metric) can be accordingly calculated (e.g., for 12 received compliant instances and 100 opportunities, this would be 12%). A general objective of the present invention is to facilitate an increase in this number. Similarly, a sub-set compliance metric can be calculated which takes into consideration for example a specific location, a user group or type of opportunity (e.g., before vs. after touching a patient). Other metrics of interest in relation to this include the number of opportunities, where, for example, an unusually high number of opportunities per time unit may indicates a busy time period, which may be a factor that also can be taken as adjusting the selector.

The mentioned sections can be implemented in any suitable fashion including the implementation by dedicated hardware, such as a computer or server, or, it can be implemented in the cloud which implies that the functions are implemented by tasks in a central or distributed processing environment. In this way, the selector 15 may have access to comprehensive knowledge of the compliance behavior in the past. This may include knowledge of one or more individuals in a group, where such a group can be defined as the cohort of staff whose behavior should be influenced in order to promote the use of hygiene equipment, and, with this, eventually reduce the damage of HAIs. The target of this embodiment of the present invention are thus individuals, e.g., staff working on a specific ward or throughout a hospital, that should be encouraged to use the provided hygiene equipment in the appropriate and desired way.

The selector can take as input measurements of individual hand hygiene events as a form of said instances. Whether an instance was compliant or not can be determined by the system or respective information can be obtained by associated meta data. For example, the information 11 can be accompanied or can be generally in a form in which it as such already can indicate a compliant instance. In such embodiments, the determination section 13 can reduce the processing to evaluating the received meta data or reception as such for determining (implicitly) the instance as compliant. Generally, an instance of using a piece of hygiene equipment is to define a specific moment or event of using a piece of hygiene equipment in comparison to the general use of such equipment. Whereas the latter use can cover a larger time span, an undefined group of users or even the mere installation of, and, with this, the possibility to use hygiene equipment, an instance can be traced to a specific event that a piece of hygiene equipment was indeed used. This may involve a specific association of that event to a particular point in time, a particular individual, and/or a particular location.

In one way, the configuration of the determination section, the selector, and the adjusting section can be seen as providing a “ticket” and associating or not associating a reward to that ticket. Each compliant hand hygiene event or “instance” can in principle generate a ticket for the selector. The adjusting section can activate or deactivate the tickets in the sense that no ticket—equaling a deactivated ticket—cannot result in a reward action in the selector. One example of such a procedure is further explained in conjunction with the below table comprising a column that indicates if the event generates a ticket or not:

| Individual# | Time | Position | Moment# | Compliant? | Ticket? |
|---|---|---|---|---|---|
| 1 | 13:52 | Bed 13:2 | 1 | 1 | Yes |
| 2 | 14:18 | Bed 8:1 | 2 | 1 | Yes |
| 1 | 14:22 | Bed 2:4 | 2 | 0 | No |
| 1 | 14:43 | Bed 3:3 | 4 | 0 | No |
| 2 | 14:58 | Bed 10:1 | 5 | 1 | Yes |

In this example one can see that individual “1” (e.g., a nurse) has generated 3 events/instances and 1 ticket, whereas another individual “2” has generated 2 events/instances and 2 tickets. The adjusting section can now influence or manipulate the probabilities that a ticket results indeed in a reward in such a way so that it promotes the desired behavioral change, for which specific examples are described as further embodiments below.

In one embodiment, the adjusting section sets the selector so that it selects equally from the compliant instances one or more instances that result in a reward action. In terms of the above-mentioned tickets this implies that each ticket has a certain chance of winning which is the same for each individual ticket.

In further embodiments, the adjusting section considers additional data associated with the received instance that can indicate an identification (ID) of an individual. In a sense, tickets of certain individuals (or combination of individuals) can be promoted by increasing their likelihood for winning in order to drive the overall reduction of HAIs in as an efficient manner as possible. This can further consider one or more of the below listed measures quantities provided and/or stored as corresponding meta data: more compliant individuals (i.e. individuals with a larger number of compliant events or tickets for promoting the as such desired behavior); less compliant individuals (encouraging the less compliant with a higher chance of winning); individuals with more frequent/total number of event/instances; individuals with less frequent/total number of events/instances (to avoid situations in which one individual generates many tickets to also have a higher chance of winning; individuals with a certain compliance rate (=usage/opportunities) over a certain time (to promote compliance rather than generating many events); individuals with a certain compliance rate change (increase) over a certain time period (encourage an improved behavior); individuals that have recently won a reward may have a decreased probability of winning. The latter may consider one or more of the following additional conditions that define a decreased probability until a certain number/proportion of his/her peers has also won, until a certain time has passed, and/or until a certain number of tickets or events has been generated by the individual or the group.

In further embodiments, the adjusting section can consider additional factors and data associated with the received instance for promoting tickets coming from certain groups of individuals (or combination of individuals/groups as per below factors) by increasing their likelihood for winning: promoting a certain professional category that can be obtained by means of ID information (i.e. increase the likelihood of a nurse winning as opposed to a doctor), promoting certain shifts and/or teams/sub-groups of the total group (e.g., night vs. day personnel, or night over day shift), promoting tickets generated in certain areas (e.g., while working in sensitive areas such as isolation units, dirty utility rooms, etc.); promoting the working with specific patients (vulnerable, difficult, etc.); promoting certain moments, situations, or procedures (e.g., “entering a bed zone”) over other moments situations, or procedures so as to focus work on tasks; promoting tickets generated at certain times of the day/week/month (e.g., hand hygiene during meal service may be important, or weekends may be promoted over weekdays).

In the above embodiments the adjusting section can thus be configured to calculate a compliance from the stored information on the past compliance, compliant instances, and/or opportunities, and to adjust the selector based on the calculated compliance. Further, the interface can be configured to receive information identifying a user operating the piece of hygiene equipment and associated with the instance and the adjusting section can be configured to calculate an individual compliance from the stored information on the past compliant instances associated with a specific user, and to adjust the selector based on the calculated individual compliance. Alternatively or additionally, the interface can be further configured to receive and/or obtain time information associated with the instance, and/or configured to receive location information associated with the instance. Yet further, the system may comprise a feedback section that is configured to store in the storage section information on an instance that resulted in a reward action.

The calculated compliance may be a figure distinct from a compliant instance, in the sense that the latter refers to an individual use event and indicates that this use was compliant. The calculated compliance, however, can be a compliance metric that is calculated from a plurality of compliant instances. For example, if the number of registered compliant instances in some given time period is relatively low, the compliance (metric) can be accordingly calculated so as to assume a relatively low value (e.g., <0.5). Likewise, a relatively high value of the calculated compliance (e.g., to be >0.5 and <=1.0) may represent a satisfactory number of registered compliant instances.

The selector which is adjustable and adjusted by the adjusting section generates a reward by selecting whether or not a given instance, e.g., in the form of a ticket, is to result in a reward action or not. In one possible implementation the selector can evaluate an expression such as

m*x>f, (1)

where m denotes a manipulation factor generated by the adjusting section dependent on any one of the factors described above, and where m>1 represents an increased chance of winning, m=1 can represent a non-manipulated ticket with the default chance of winning, and m<1 can be a decreased chance of winning. A special case can be m=0 when even compliant behavior is not eligible for a win (this could for instance be used to block a recent winner from winning immediately again). Likewise m=infinity is the other extreme where a certain case always will win.

Further, in the above expression (1) x can denote a random number between 0 and 1, which may be generated from a standard uniform distribution. In the cases where x has a normal distribution can of course be considered, but may render the selection more complex and less transparent also for the administrator and operator, who—ultimately and in line with the embodiments of the present invention—wishes to adjust the system for a well-defined target purpose, namely the appropriate and effective promotion of the proper use of hygiene equipment.

Further, in the above expression (1) f can denote a constant, adjusting the frequency with which rewards are given as a whole. For example, if m=1 then f=0.95 indicates a chance of 5% for any ticket to win. In a practical example a ward can generate, e.g., 1,000 events/day and—accordingly—f=0.9990 will mean that on average one reward will be given per day (if all events were compliant). A special case may occur if f>1 in which case only promoted instances are eligible for a reward, and not “ordinary” compliant behavior.

In another embodiment, the selector may work on the basis of the expression

m1*(x+m2)+m3>f, (2)

where m1 is basically the same as the above m of expression (1), and m2 and m3 can be seen as “floor”-levels that can be manipulated to add a constant, non-random manipulation factor which can be either scalable with the main factor m1 or independent of it (m2 and m3, respectively). Further, linear and non-linear equations can contribute to expression ultimately operating the adjustable selector.

In general, the reward action as such can be or result in anything that is appreciated by the individuals, ranging from cup-cakes to diamond rings. Practically the reward can be distributed as vouchers sent out via e-mail or text message, or the reward can also be formally and physically handed out during, for example, a ward-meeting. Further examples for rewards and reward actions are provided in conjunction with the applicable embodiments as described elsewhere in the present disclosure.

FIG. 1B shows a schematic view of a monitoring system for hygiene equipment according to a general embodiment of the present invention. Some features and elements of the shown system 1′ are similar or identical to those as described in conjunction with FIG. 1A and are accordingly denoted by same reference numerals. In this embodiment, however, the system 1′ comprises a further interface 18 that is configured to receive information 19 indicating an opportunity to use a piece of hygiene equipment. In another related embodiment, the functionalities of the further interface 18 are combined with the functionalities of the interface 12 so that one interface is configured to receive or obtain information on both the use instances of a piece of hygiene equipment and an opportunity to use the piece of hygiene equipment.

In such embodiments a determination section 13 can compare the information 11 received for a use of a piece of hygiene equipment to the information 19 received for an opportunity to use the piece of hygiene equipment for determining whether or not the use is actually compliant or not. For example, only a use instance which has a corresponding opportunity may be determined as compliant so as to reliably promote the correct, i.e. compliant behavior. Further, such embodiments may avoid abuse of the system, by making it difficult to obtain a reward by merely operating hygiene equipment as often as possible.

This may increase the number of use instances but may not reflect any appropriate and compliant use thereof.

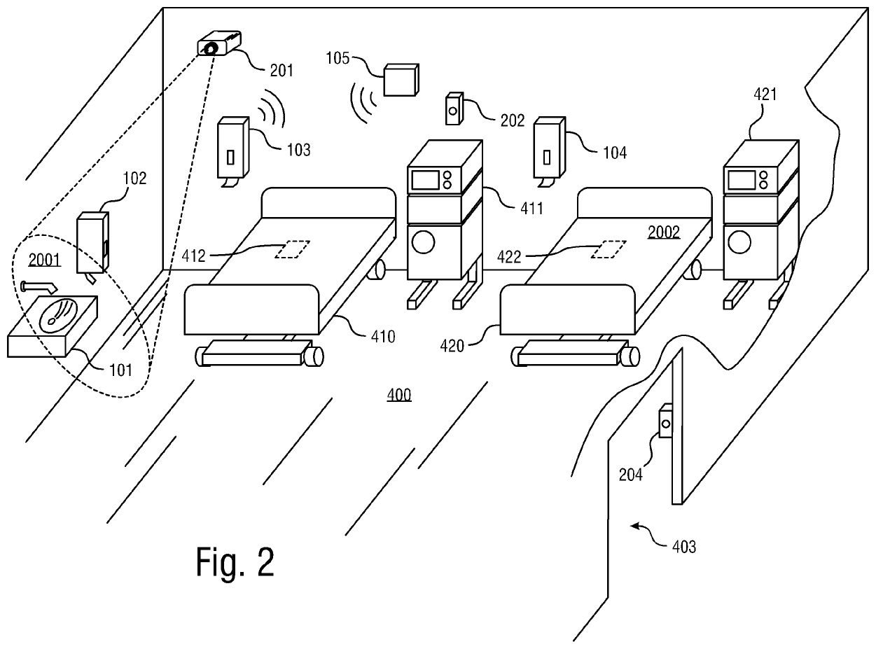

FIG. 2 shows a schematic view of a deployment example of an embodiment of the present invention. As an example only, there is shown as a facility an intensive care unit 400 with corresponding intensive care points: first and second patient stations 410, 420 and first and second patient care equipment 411, 421. As can be seen, the intensive care unit 400 may be occupied by one or two patients in the shown configuration. However, further embodiments of the present invention may envisage also other intensive care units with any number of patients and personnel and/or other facilities such as stationary care, retirement or care homes, private practices, hotels, etc. Herein, the usage of pieces of hygiene equipment is endorsed and in accordance to, e.g., the World Health Organization (WHO) recommended “My 5 Moments Of Hand Hygiene”. As such, a detection of using a piece of hygiene equipment, based on an opportunity to use the piece of hygiene equipment constitutes a compliant behavior.

The opportunity data is obtained through receiving signals from one or more sensors that may be distributed in the intensive care unit 400. For example, opportunity data is obtained through receiving signals from pressure (or heat) sensors 412, 422. If one or more of such sensor signals indicate that a corresponding patient station 410, 420 is in use, the opportunities for the use of hygiene equipment will accordingly change. For example, an algorithm may correlate signals from the vicinity sensor 202 and the sensors 412, 422, in a way that signals that indicate an activation of the vicinity sensor 202 (i.e. operator present) and a first pressure sensor 412 correlate to opportunities only in connection with the first patient station 410, whilst signals that indicate an activation of the vicinity sensor 202 and the first pressure sensor 412 and the second pressure sensor 422 correlate to opportunities in connection with both patient stations 410, 420.

Also, an opportunity sensor arrangement may be provided in the intensive care unit 400 that comprises one or more cameras 201, one or more vicinity sensors 202, and one or more door passing sensors 204. In the example of FIG. 2, the camera 201 may be in a particular arrangement for detecting an opportunity in a dedicated area, such as the surrounding area 2001 of the hand wash sink 101. For example, the image and/or video data obtained from the camera 201 may be processed or analyzed for determining whether an individual could/should have used the soap dispenser 102 when washing hands at sink 101. In a way, the use of the sink 101 implies also an opportunity to use the soap dispenser 102. Such visual determination of an opportunity by images and/or a human observer may also be selected for opportunities that are difficult to detect by sensors, such as a healthcare worker performing an aseptic task (WHO moment Nr. 2) or after a body fluid exposure risk (WHO moment Nr. 3). It may be preferable to further consider that WHO moments Nr.s 1 & 5 may be correlated to moments 2 & 3, which can be exploited for removing sensors that either sense moments 1 & 5 or 2 & 3 in the second opportunity sensor arrangement. Such correlations may also be made automatically by the machine learning algorithm.

Likewise, a vicinity sensor 202 may determine the opportunity to use the second disinfectant dispenser 104 when an individual operates first patient care equipment 411 which, in turn, can indicate that manual operations or actions are carried out to patient in first patient station 410, which can be, for example, a bed. In general, any one of the equipment sensors and opportunity sensors may convey signals in any suitable manner, such as by wire-bound communication or wireless communication as, for example, shown between the first disinfectant dispenser 103 and a wireless data acquisition and collection point 105.

In the configuration shown in FIG. 2 usage data is obtained either directly from an equipment sensor arrangement provided for one or more of the individual pieces of hygiene equipment, such as the soap dispenser 102, the first and second disinfectant dispensers 103, 104, and the hand washing sink 101, or indirectly by use of one or more cameras 201. In this way, the system is able to receive usage data from these pieces of equipment 101-104 as possibly individual signals from each corresponding device/sensor.

An instance of using a piece of hygiene equipment, based on the opportunity of using the piece of hygiene equipment, is hereon referred to as a compliant instance. Based on a history of usage instances, the reward hygiene system is able to compute a compliance metric, which will be explained in more detail later. Also, and possibly based on the compliance metric, the reward hygiene system selects a compliant instance to result in a reward action, which is explained in more detail later. Therefore, the possibility of such a reward action is motivation for the personnel active in the intensive care unit 400 to use a piece of hygiene equipment 101-104 in a compliant manner. Merely selecting a usage instance, i.e. compliant and non-compliant instances, to result in a reward action may lead the personnel to repetitively and unnecessarily use hygiene equipment. Therefore, compliant behavior is only motivated when the possibility of resulting in a reward action is based on a compliant instance.

The configuration as shown in FIG. 2 is only to be seen as an application example and the embodiments of the present inventions may well be applicable also to configurations different from the environment of an intensive care unit 400 in particular, or from a hospital in general. In a way, a closed loop is provided when it is considered that upon usage of the hygiene equipment, this use is detected by respective sensors and fed to the reward hygiene system, where the received usage data is assessed and evaluated with regard to usage opportunity, resulting in a possibility of a reward action, incentivizing the personnel to use the hygiene equipment in a compliant manner.

In a sense, some embodiments of the present invention may provide a closed feedback loop for obtaining an increased compliance by the individuals and by considering the group of individuals as the system to be subject to control and the an embodiment of the disclosed systems as the control system:

| | |
|---|---|
| INITIAL | |
| BEHAVIOR | → HYGIENE EQUIPMENT → SENSOR → |
| | → USE INSTANCE → COMPLIANCE → |
| | → PAST BEHAVIOR → REWARD ACTION → |
| IMPROVED | |
| BEHAVIOR | |

In other words, an individual initially uses the hygiene equipment in an unsatisfactory manner, e.g., by not using it at the right moments (i.e. compliant) or not at all. Through one compliant use the individual has the possibility to obtain a reward action. As a result, the individual is encouraged and rewarded and shows an improved behavior as compared to the initial behavior.

In further embodiments, there may be provided several “levels” of rewards, meaning that a relatively minor reward (e.g., a cup-cake) may be given relatively frequently and that a relatively more expensive reward (e.g., a diamond ring is also possible but significantly less frequent. This can serve the purpose of constantly reminding users via winning a relatively cheap reward that also the winning of a relatively expensive reward is possible. With this an additional incentive can be obtained so as to help the promoting of the use of hygiene equipment. An implementation can consider setting the already mentioned parameters to, for example, f>0.95 for a relatively cheap reward and to, for example, f>0.99999 for a relatively expensive reward. Also intermediate levels may be provided to implement a finer approach.

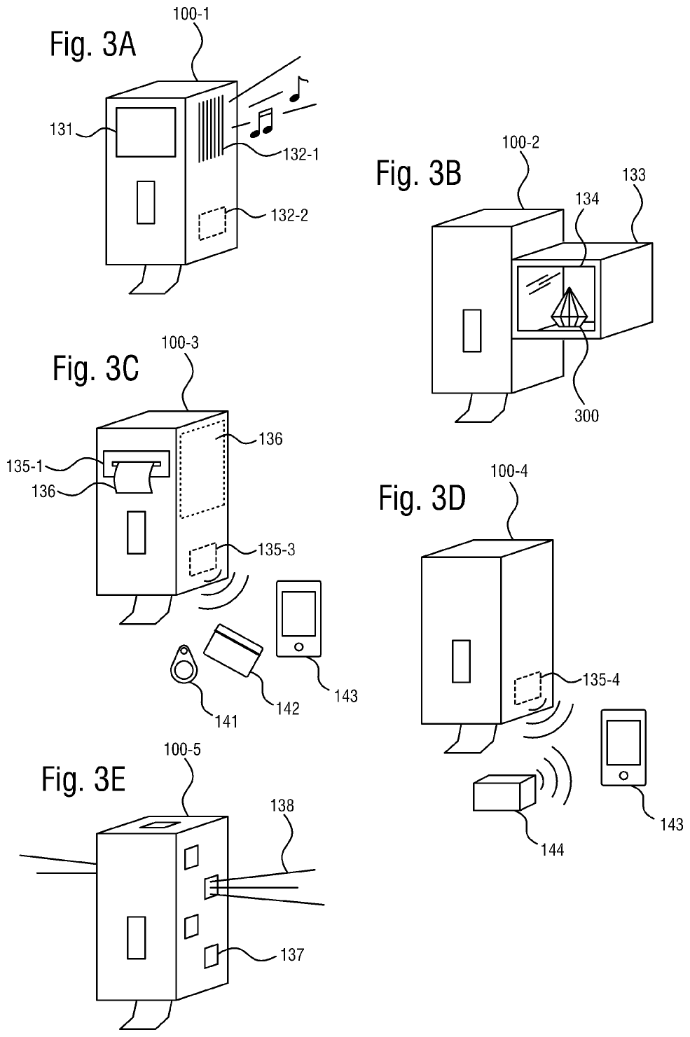

FIGS. 3A to 3E show schematic views of pieces of hygiene equipment according to corresponding further embodiments of the present invention demonstrating various but exemplary ways for implementing a reward action. As shown in FIG. 3A, a piece of hygiene equipment 100-1 comprises a display 131 as well as an audio output 132-1. At the same time, the piece of hygiene equipment 100-1 may comprise a corresponding section for generating a display output to be output on display 131 as well as a sound output to be output by the audio output 132-1. As far as the latter sound is concerned, possible examples include acoustic signals, sound jingles, songs, music, speech, and the like and any combination of that, and the reward action will comprise controlling the output of corresponding sound signals. The audio output 132-1 comprises a speaker or a piezo-type sound emitter and corresponding driving circuitry.

The piece of hygiene equipment 100-1 may further comprise a code generation section 132-2 configured to generate a code as a reward token, that can be used as a voucher for making online purchases, free theatre or cinema tickets, free access to gyms and/or spas, and/or free consumption in a cafeteria and/or restaurant of, for example, the very premises at where the piece of hygiene equipment is installed. For example, a code may be displayed as some sort of reward action for obtaining a free beverage at the hospital's cafeteria. The displayed code can be a human readable alphanumeric code or a one- or two-dimensional barcode (e.g., QR code) so that it can be read by, for example, a smartphone.

The generated code may generally represent a value token and should be generated so as to avoid unauthorized entities from generating or obtaining valid codes. This may be achieved by, for example, generating and optionally also encrypting codes ad hoc using a local key stored in the piece of hygiene equipment, obtaining codes from a list of pre-generated codes stored on the piece of hygiene equipment, or receiving codes from an external entity. Thus, on the display 131 a reward action can be displayed as form of a code generated by the code generation section.

FIG. 3B shows a further piece of hygiene equipment 100-2 which is provided with a closed compartment 133 (enclosure), which can accommodate a physical object 300 as a reward. Preferably, the enclosure 133 is equipped with a transparent door 134 and/or sidewall so that an operator may well see the reward object 300. Such objects may include gifts or giveaways, product samples, physical cinema or theatre tickets, physical vouchers, and the like. The reward action in this case will comprise an operation of opening the door 134 or, in more general terms, releasing the object 300 to an operator. This may include the driving of magnetic or electric actuators and/or motors.

FIG. 3C shows a further example of a piece of hygiene equipment 100-3. The shown piece of hygiene equipment comprises a printer 135-1 which is arranged to print out a slip 136 which can be, in turn, a voucher, ticket, lottery ticket, or the like. In this context, the reward action 18 will be the operation of the printer 135-1 which will be in turn the production of the output slip 136. The printed slip 136 may display a code. If the piece of hygiene equipment 100-3 comprises a code generation section as the device 100-1 of FIG. 3A, the code may be generated by a code generation section 135-2 of the piece of hygiene equipment, as the reward action 18. The device 100-3 may further include an operator detection section 135-3 that is configured to determine a property of an operator using the hygiene equipment 100-3.

For example, in this specific embodiment, the operator detection section 135-3 comprises a operator detection sensor (e.g., near field communication receiver, Bluetooth™ receiver, RF-tag sensor, one- or two-dimensional barcode reader, etc.) shown on FIG. 3C, and may detect from a token 141, a card or badge 142, or a mobile device 143 (e.g., a mobile phone, a tablet, a pager), etc. carried by the operator a property of the operator so as to be able to distinguish at least between two individuals, persons, or groups. The determined property of the operator may be information identifying an operator to be rewarded, or information identifying a group of persons to which the operator belongs. For example, in some situations one individual/group may be selected to be rewarded by a reward action 18, whilst another one is not. For example, hospital staff (to be rewarded) can be distinguished from visitors (not to be rewarded), and employees can be distinguished from management, etc. The adjustable selector section 17 may, in such situations, be further configured to suppress a reward action 18 based on the determined property.

Thus, the property of the operator may be used to determine the control command, to control the reward action 18, to generate the code, or to forward the code toward the operator. Instead of, or in addition to the sensor, the operator detection section 135-3 may comprise an input device and the operator may input information from which the operator property may be determined. The input device may be any known input device such as a keypad, a keyboard, a touch screen, etc. For example, the operator may input information identifying him- or her, such as an employee ID number, a name, a mobile telephone number, an e-mail address, etc. However, the sensor and/or input device may be external to the device 100-3, and the operator detection section 135-3 may be configured to obtain information from these to determine the property of the operator. Although shown in conjunction with FIG. 3C, the above operator detection section 135-3 and the corresponding configuration of the adjustable selector section 17 may be well combined with any other embodiment of the present invention.

FIG. 3D shows a further piece of hygiene equipment 100-4 which comprises an operator detection section 135-4 obtaining, as the property of the operator, identification information indicating an operator to be rewarded such as an employee ID number, a name, a telephone number, an e-mail address, identification information of a communication or social network (e.g., a device MAC address, a twitter handle, a URL of a profile, etc.) or any other information identifying the operator, or a group of persons to which the operator belongs. For this purpose a communication section may be employed that communicates with a device 143, 144 for retrieving the property of the operator. For example, a phone 143 may communicate any mentioned ID to the piece of hygiene equipment 100-4. Said communication section may further be configured to forward a code toward the operator, based on the obtained operator identification information, so that the code may be used by the operator.

For example, the code may be generated by a code generation section in the piece of hygiene equipment 100-4 and forwarded directly toward the operator by the communication section or it may be forwarded to an external device 144, such as a server which in turn stores the code and/or forwards it to the operator, or a machine delivering rewards upon use by the operator. For example, the piece of hygiene equipment 100-4 may forward a code to a vending machine, and when the operator having the determined property uses the vending machine, a reward may be delivered.

Alternatively, the reward action 18 may simply be a transmission of information to the server, and the server may generate a code instead. The piece of hygiene equipment 100-4 or the server, as the case may be, may determine how to forward the code to the operator based on the determined property of the operator. For example, if the property of the operator is an email address, the server may cause an email including the code to be transmitted toward the determined email address. As another example, if a property of the operator is an indication that the operator belongs to a certain group of persons, the server may be configured to store the code on a database, in association with the group to which the operator belongs.

In any case, forwarding the code to an operator may encourage desired habits by the operator, when receiving the code or when using the code to obtain a reward. By generating the code and providing it directly to the operator (e.g., by displaying it, or printing it on a slip), the operator may be more comfortable using the piece of hygiene equipment 100-4, knowing that no information which may identify him or her would be shared with external entities. Similar effects may be achieved by embodiments forwarding the code directly to the operator.

Alternatively, forwarding the code to an external device 144 such as a server storing codes in association with information identifying operators may be used to reinforce positive group reinforcement. The codes generated for a group of operators may be “pooled” and used together to reward a whole group of operators, instead of, or in addition to individual rewards. For example, a shift of hospital workers may decide to group the codes they each receive in a pool.

Codes may allow monitoring of the compliance to hygiene rules by groups of people without causing individual operators to feel personally targeted, if the codes are not associated with individuals but with groups of operators. In addition, pools of codes may be used to create competition between groups of operators by forming contests between different groups, or by offering better rewards when the compliance level of a group is high, to further increase motivation for hygiene compliance.

FIG. 3E then shows a further piece of hygiene equipment 100-5 which is provided with light signal emitting elements 137 that are configured to emit various light signals 138 of various colors and flashing patterns. In this embodiment, the reward action will be the operation of the light-emitting elements 137 so as to emit a more or less predetermined light pattern comprising the light flashes 138. This example may be preferable in the sense that not only the individual operator using the equipment 100-5 becomes aware of the reward action but also individuals in the near vicinity can take note of the fact that a reward is taking place.

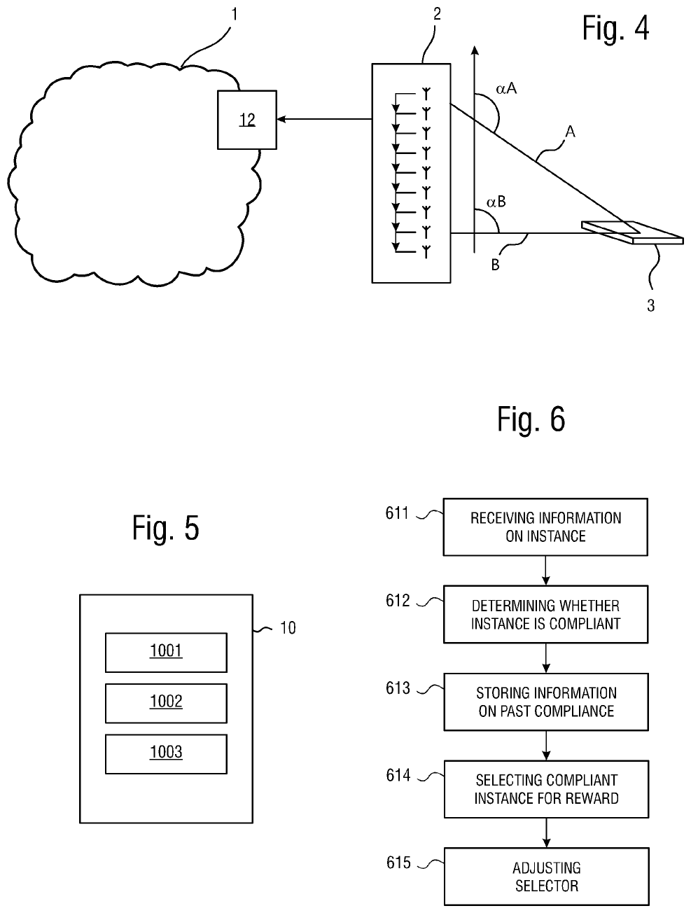

FIG. 4 shows a schematic view of a system coupled to an indoor positioning system according to another embodiment of the present invention. In this embodiment the system's interface 12 (and/or 19) is coupled to a positioning system 2 that is configured to obtain and/or process position data of one or more individuals using a piece of hygiene equipment. The positioning system 2 can be an indoor positioning system that is configured to obtain and/or process position data of the one or more individuals by obtaining angular information on signals being received from said one or more individuals.

Specifically, the indoor positioning system 2 can receive signals from a transmitter 3 from different directions by means of an antenna array. The transmitter 3 may be a tag, a device, a radio or telephone device, or any other suitable device that is carried by an individual who is supposed to use hygiene equipment as subject to monitoring by a system according to an embodiment of the present invention. The technology of indoor positioning is as such known and may be specifically referred to as AoA standing for angle of arrival. Likewise, however, also other positioning systems can be applied for the purposes of the present invention such as time of flight systems, including GPS, Gallileo, Glonass, and also other local, low-range systems.

In such embodiments, the position of the individuals can be tracked for determining a use instance and/or an opportunity. For example, the positioning system may detect an individual in a dedicated vicinity of a piece of hygiene equipment and can thus assume an instance of using this piece of hygiene equipment. Likewise, the positioning system may detect an individual entering a vicinity of a patient or bed and may thus determine an opportunity to use or to have used a piece of hygiene equipment accordingly.

FIG. 5 shows a schematic view of a general apparatus embodiment of the present invention. A corresponding apparatus, e.g., a computer, server, or—generally—any processing resource of, e.g., a data center or a cloud computing environment comprises or has access to a processing unit 1001, a memory unit 1002 and a communication unit 1003. The memory unit 1002 stores code for the processing unit 1001 to be executed, and, thereby implementing any embodiment as part of the present disclosure.

FIG. 6 shows a schematic view of a general method embodiment of the present invention. Accordingly, there is provided a method for operating a system that monitors hygiene equipment. The general method embodiment at least comprises the following: A step 611 of receiving information indicating an instance of using a piece of hygiene equipment, a step 612 of determining whether the instance is a compliant instance to use the piece of hygiene equipment, a step 613 of storing information on past compliance, a step 614 of selecting with an selector whether the compliant instance is to result in a reward action, and a step 615 of adjusting the selector based on the stored information on the past compliance.

Although detailed embodiments have been described, these only serve to provide a better understanding of the invention defined by the claims and are not to be seen as limiting.

1. A monitoring system for hygiene equipment, the system comprising:
an interface configured to receive information indicating an instance of using a piece of hygiene equipment;
a determination section configured to determine whether the instance is a compliant instance to use the piece of hygiene equipment;
a storage section configured to store information on past compliance;
a selector configured to select whether the compliant instance is to result in a reward action; and
an adjusting section configured to adjust the selector based on the stored information on the past compliance.

2. The monitoring system according to claim 1, further comprising a processing section configured to amend the stored information on past compliance.

3. The monitoring system according to claim 2, wherein the processing section is configured to amend the stored information on past compliance based on one or more received instances.

4. The monitoring system according to claim 3, wherein the interface is further configured to receive information indicating an opportunity to use the piece of hygiene equipment, and wherein the determination section is configured to determine whether the instance is a compliant instance by matching an instance of using and an opportunity.

5. The monitoring system according to claim 4, wherein the processing section is configured to amend the stored information on past compliance based the received information indicating an opportunity.

6. The monitoring system according to claim 1, wherein the interface is coupled to a positioning system that is configured to obtain and/or process position data of one or more individuals using a piece of hygiene equipment.

7. The monitoring system according to claim 6, comprising an indoor positioning system that is configured to obtain and/or process position data of the one or more individuals by obtaining angular information on signals being received from said one or more individuals.

8. The monitoring system according to claim 1, wherein the adjusting section is configured to calculate a compliance from the stored information on the past compliant instances, and to adjust the selector based on the calculated compliance.

9. The monitoring system according to claim 1, wherein the interface is further configured to receive information identifying a user operating the piece of hygiene equipment and associated with the instance.

10. The monitoring system according to claim 9, wherein the adjusting section is configured to calculate an individual compliance from the stored information on the past compliant instances associated with a specific user, and to adjust the selector based on the calculated individual compliance.

11. The monitoring system according to claim 1, wherein the interface is further configured to receive and/or obtain time information associated with the instance.

12. The monitoring system according to claim 1, wherein the interface is further configured to receive location information associated with the instance.

13. The monitoring system according to claim 1, further comprising a feedback section configured to store in the storage section information on an instance that resulted in a reward action.

14. The monitoring system according to claim 1, wherein a piece of said hygiene equipment is any one of a soap dispenser, a towel dispenser, a disinfectant dispenser, an alcogel dispenser, a tissue dispenser, a hygiene article dispenser, a waste bin, a used towel bin, and a toilet paper dispenser.

15. A method for operating a system that monitors hygiene equipment comprising the steps of:
receiving information indicating an instance of using a piece of hygiene equipment;
determining whether the instance is a compliant instance to use the piece of hygiene equipment;
storing information on past compliance;
selecting with an selector whether the compliant instance is to result in a reward action; and a step of
adjusting the selector based on the stored information on the past compliance.
